(19) United States
(12) Patent Application Publication
(10) Pub. No.: US 2008/0234586 A1
(43) Pub. Date: Sep. 25, 2008

(54) SYSTEM AND METHOD FOR PROVIDING NONINVASIVE DIAGNOSIS OF COMPARTMENT SYNDROME USING EXEMPLARY LASER SPECKLE IMAGING PROCEDURE

(75) Inventors: Guillermo J. Tearney, Cambridge, MA (US); George Velmahos, Dover, MA (US); Brett E. Bouma, Quincy, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 12/051,600

(22) Filed: Mar. 19, 2008

Related U.S. Application Data
(60) Provisional application No. 60/895,642, filed on Mar. 19, 2007.

(51) Int. Cl. A61B 5/02

(52) U.S. Cl. 600/479

(57) ABSTRACT

Exemplary systems and methods can be provided for providing information associated with tissue. For example, it is possible to illuminate the tissue with at least one electromagnetic radiation which is a coherent light and/or a partially coherent light. The electromagnetic radiation reflected from the tissue can be received and speckle patterns may be formed associated with the electromagnetic radiation. In addition, changes can be analyzed in the speckle patterns at time intervals sufficient to measure motion of or within a fascial compartment of the tissue. For example, it is also possible that the electromagnetic radiation is an interfered radiation from a sample and a reference. Further, the speckle patterns can be measured at different depths within the sample by moving the reference.

### CROSS-REFERENCE TO RELATED APPLICATION(S)

The present invention claims priority from U.S. patent application Ser. No. 60/895,642 filed on Mar. 19, 2007, the entire disclosure of which incorporated herein by reference.

### FIELD OF THE INVENTION

The present invention relates to system and method which can utilize a noninvasive optical device capable of detecting extremity compartment syndrome.

### BACKGROUND INFORMATION

Extremity compartment syndrome is believed to be a major cause of morbidity and limb loss following civilian and military trauma. In modern warfare, the combination of new explosive devices and more effective protection of the torso makes injuries to the extremities a primary concern. Abdominal compartment syndrome is also a concern in patients with trauma. In the early phases of compartment syndrome, an increased interstitial pressure within enclosed fascial compartments likely decreases capillary arteriovenous pressure gradients, slowing capillary blood flow. Cessation of capillary blood flow results in edema which further increases interstitial pressure, eventually leading to ischemia and permanent damage to the muscles and nerves. Early identification of the developing compartment syndrome is crucial in order to offer therapeutic interventions in a timely manner.

Unfortunately, the unreliability of clinical symptoms—particularly in multiply injured patients—and the lack of accurate diagnostic techniques often lead to delayed diagnosis and interventions with disastrous outcomes. Presently, the only accepted method for measuring compartment pressure is invasive and requires insertion of the device into a body cavity or fascial compartment. It would be preferable to have system and method for monitoring intra-compartmental pressures that was non-invasive.

### OBJECTS AND SUMMARY OF EXEMPLARY EMBODIMENTS OF THE INVENTION

Exemplary objects of the present invention may include, but not limited to the detection of blood within compartments, detecting motion and blood flow below the skin, and validating (e.g., in humans at risk of compartment syndrome.

Detection of motion and blood flow within compartments The exemplary embodiments of the methods and systems according to the present invention described herein can be utilized to measure blood flow in fascial or abdominal compartments. A further exemplary embodiment can quantitatively determine the distributions of blood flow in compartments. An additional exemplary embodiment determines the presence, absence, or degree of capillary blood flow in compartments. Another exemplary embodiment can determine the pressure in fascial or abdominal compartments by measuring blood flow or Brownian motion or a combination thereof.

Detection of motion and blood flow below the skin An exemplary embodiment of the system and method according to the present invention can be provided that measures the motion or blood flow of internal structures while at least partially discriminating between skin blood flow and internal structures. A further exemplary embodiment can include systems and methods for obviating skin blood flow so that internal motion or blood flow can be determined.

Validation in humans at risk of compartment syndrome An exemplary embodiment of the system and method according to the present invention can be compared to the conventional invasive systems and methods of measuring compartment pressures (e.g., Stryker® compartment pressure monitor) in patients who are at risk for the syndrome and monitored per standard of care with frequent measurements.

Since the early stages of compartment syndrome can change muscle capillary blood flow, according to one exemplary embodiment of the present invention, it has been believed that the detection of capillary blood flow within fascial compartments can provide an index for predicting compartment syndrome. Using such exemplary embodiment, a noninvasive method and system [e.g., termed Laser Speckle Imaging (LSI)] can be provided for measuring a depth-dependent tissue perfusion of skin. With the exemplary LSI methods and systems, coherent light can illuminate the tissue, may multiply scattered within the tissue, and can be remitted to form a speckle pattern that is imaged at the surface and analyzed spatially and temporally.

The exemplary LSI methods and systems may be capable of measuring blood flow since speckle formed from light that has traversed vascular tissue is temporally modulated, where the modulation frequency can dependent on blood flow rate. Due to optical diffusion, spatial analysis of the speckle pattern allows measurement of blood flow distributions at different depths within tissue. Exemplary advantages of these exemplary LSI methods and systems for measuring tissue perfusion can include noninvasiveness, have the potential for portability, and may be relatively low cost. Further, according to another exemplary embodiment of the present invention, it is possible to utilize a hand held device with a simple interface that can indicate high or low risk of compartment syndrome, and which may not need significant training to interpret.

One difficulty with utilizing the LSI method and system for measuring internal blood flow can be that the skin blood flow also can modulate the laser speckle pattern, making it difficult to measure the components of the laser speckle modulation that may be due to motion or flow in a fascial compartment or internal body cavity such as the abdominal cavity, peritoneum or pleural cavity. One exemplary method for obviating external (e.g., skin) flow can be to apply a tourniquet to the skin or external member in a manner such that the external flow is substantially diminished, so that the LSI measurement primarily only reflects the motion or flow of the internal member, cavity, or fascial compartment. Another exemplary embodiment of system and method according to the present invention can be provided that may apply local pressure at the measurement site, thereby substantially terminating blood flow or motion in the measurement area. Thus, with such exemplary embodiment, it possible to measure the subsurface motion or flow using laser speckle pattern modulation measurement of the internal cavity or fascial compartment below the skin.

Accordingly, exemplary systems and methods can be provided for providing information associated with tissue. For example, it is possible to illuminate the tissue with at least one electromagnetic radiation which is a coherent light and/or a partially coherent light. The electromagnetic radiation reflected from the tissue can be received and speckle patterns may be formed associated with the electromagnetic radiation. In addition, changes can be analyzed in the speckle patterns at time intervals sufficient to measure motion of or within a fascial compartment of the tissue. For example, it is also possible that the electromagnetic radiation is an interfered radiation from a sample and a reference. Further, the speckle patterns can be measured at different depths within the sample by moving the reference.

According to one exemplary embodiment of the present invention, the motion can include blood flow, and the blood flow may be capillary blood flow. It is also possible to receive the electromagnetic radiation upon an application of pressure to or at a distance from the tissue. The tissue can be illuminated at a location position, and the electromagnetic radiation can be received at a second location. The first and second locations are separated from one another by a predetermined distance. According to still another exemplary embodiment of the present invention, it is possible to provide various arrangements which perform the above-described exemplary techniques in a hand-held arrangement.

These and other objects, features and advantages of the present invention will become apparent upon reading the following detailed description of embodiments of the present invention..

### BRIEF DESCRIPTION OF DRAWINGS

Further objects, features and advantages of the invention will become apparent from the following detailed description taken in conjunction with the accompanying figures showing illustrative embodiments of the invention, in which:

FIG. 1 is a schematic block diagram of an exemplary embodiment of a LSI system for measuring compartment flow according to the present invention;

FIG. 2 is an exemplary image of laser speckle remitted from tissue;

FIG. 3 is a diagram illustrating an exemplary measurement of spatial variations in speckle pattern modulation;

FIG. 4 is an illustration of an exemplary implementation of an exemplary embodiment of a processing method for temporal analysis of time integrated laser speckle patterns according to the present invention;

FIG. 5 is an exemplary graph showing an exemplary relationship between time integrated speckle pattern measurement of phantom (Teflon) velocity versus true velocity;

FIG. 6 is an exemplary photograph of laser speckle from an arm with a measurement distance that is far from the beam illumination location; and

FIG. 7 is a block diagram of an exemplary embodiment of a low coherence laser speckle pattern measurement device according to the present invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

According to one exemplary embodiment of the present invention, it is possible to use the exemplary LSI systems and methods for measuring the depth-dependent tissue perfusion, as described herein.

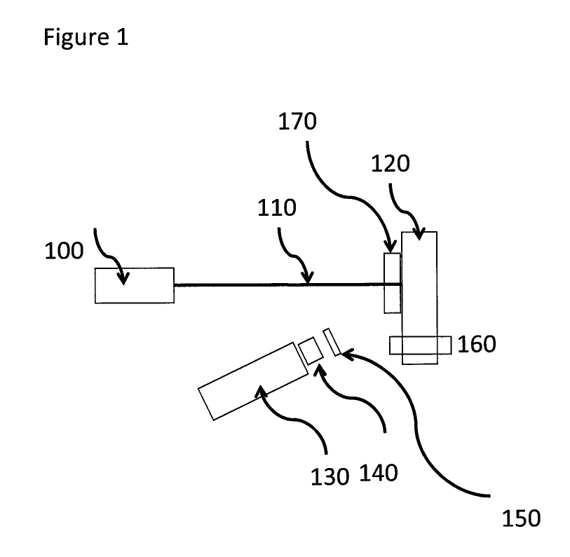

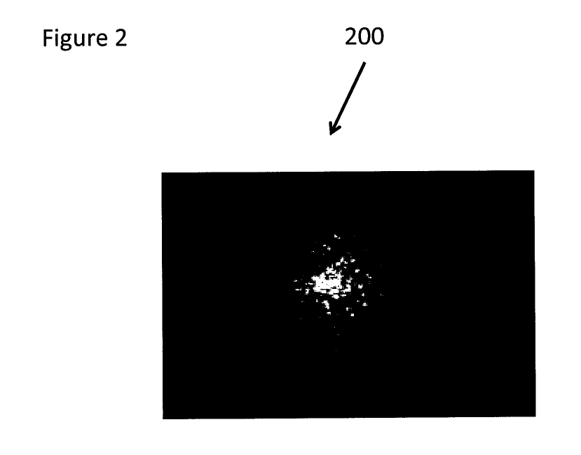

Development and Validation of Exemplary LSI System and Method. As shown in FIG. 1, an exemplary embodiment of the LSI system according to the present invention can be provided which may use a HeNe 100 laser to illuminate tissue 120 with a light 110 (or other types of electromagnetic radiation) and a high-speed CCD or CMOS camera 130. Such exemplary CCD or CMOS camera 130 can image the speckle pattern remitted from tissue such as an arm, leg or abdominal cavity 120 through a lens 140 and a polarizer 150 that is cross polarized with respect to the illumination light 110. In one exemplary embodiment, the CCD or CMOS camera 130 is capable of acquiring images at frame rates of about 1000/s, e.g., for detecting a remitted laser speckle patterns 200 (shown in FIG. 2) as a function of time.

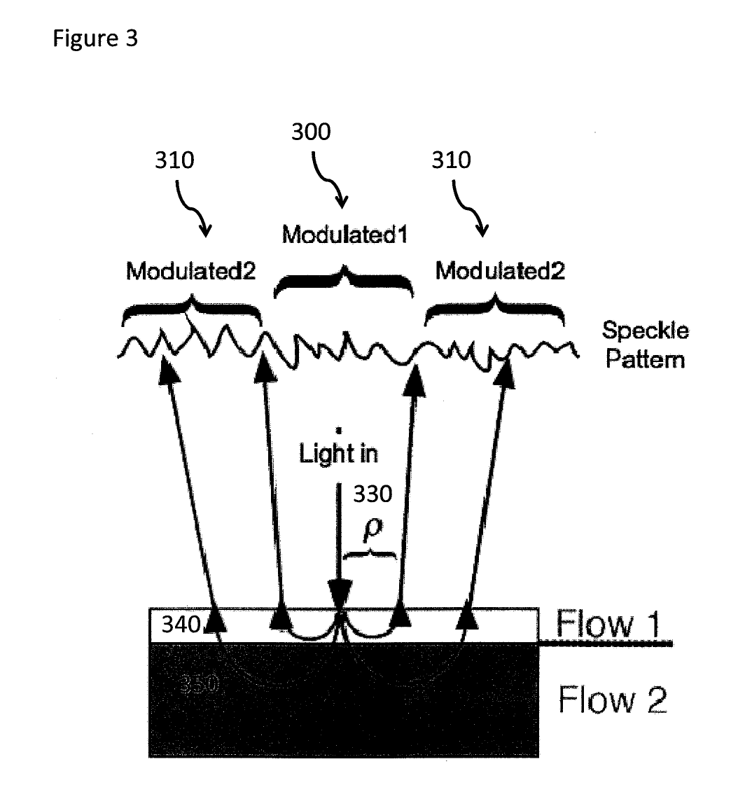

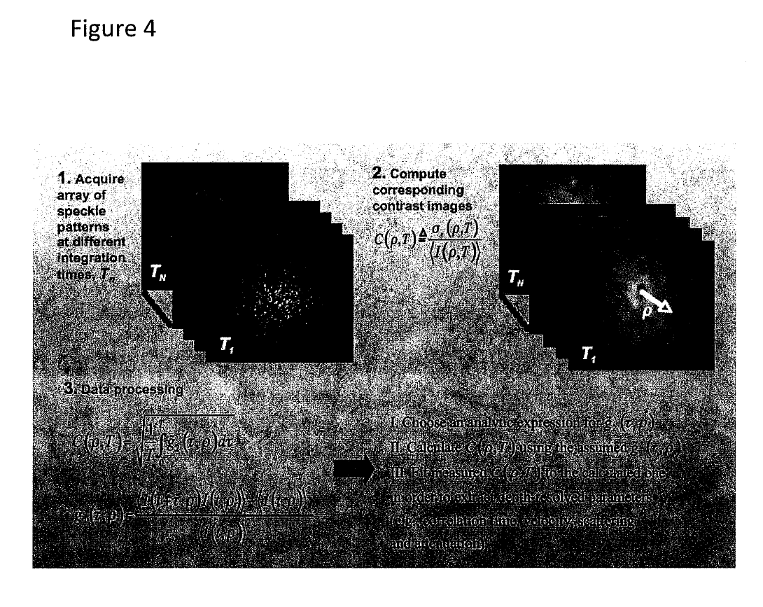

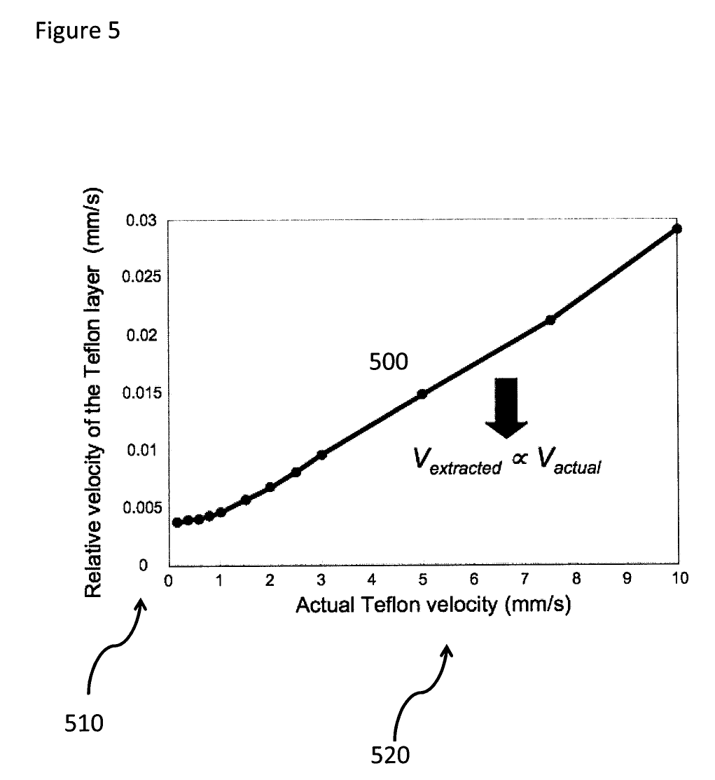

It is also possible to utilize software to analyze the temporal 300 and spatial modulation 310 of laser speckle patterns in order to extract depth-dependent blood flow distributions, as shown in FIG. 3 which illustrates an exemplary measurement of spatial variations in speckle pattern modulation. For example, FIG. 4 shows an illustration of an exemplary implementation of an exemplary embodiment of a processing method for temporal analysis of time integrated laser speckle patterns according to the present invention. Using such exemplary implementation, a linear relationship 500 can be observed between the velocities 510 of scattering phantoms (about 0.2-10 mm/s), measured by the exemplary LSI system and method and the true velocities of the phantoms 520 (e.g., R=0.99, p<0.001), likely indicating that the exemplary LSI methods and systems may be utilized to recover flow velocity, as provided in FIG. 5.

Development and Validation of Exemplary Method for Depth-Resolved Flow Estimation. The exemplary embodiment of the system and method according to the present invention can be used to determine the speckle pattern decorrelation as a function of distance from the illumination point, and may fit the correlation curves to multiple exponential functions. To establish the exemplary relationship between the exponential decay constants and photon penetration depth, it is possible to utilize a diffusion theory to fit the long-time integrated speckle pattern to the optical properties (e.g., μₛ, μₐ, and g) of the tissue. These exemplary optical properties can be input into, e.g., a Monte Carlo model to determine the relationship between the radially-dependent decay constants (e.g., flow distributions) and depth. Such exemplary embodiment of the method and system according to the present invention can be using in human tissue, and a high correlation can be obtained between thicknesses of two layers with different velocities measured by the exemplary LSI method, system and histology.

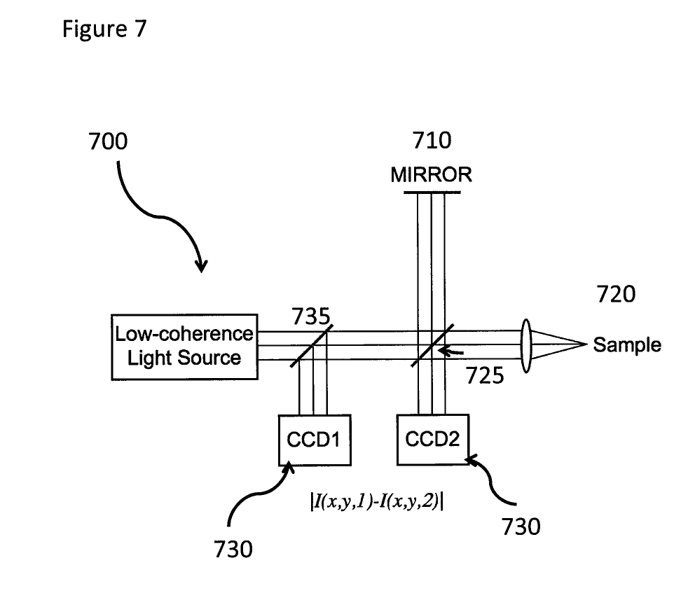

It is possible to use the exemplary embodiments of the LSI system and method according to the present invention for providing a compartment syndrome diagnosis that can include measuring much deeper into the tissue to probe muscle capillary flow and avoiding confounding speckle modulation caused by blood flow in the skin. It is also possible to utilize exemplary variants of the exemplary LSI system and method that can be optimized to observe deep capillary tissue perfusion noninvasively by, e.g., a) using longer wavelengths (e.g., 1.3 μm) to increase tissue penetration, b) optimizing the imaging geometry to maximize penetration, and c) investigating low-coherence speckle interferometry to measure LSI patterns that only result from relevant tissue scattering path lengths (FIG. 7).

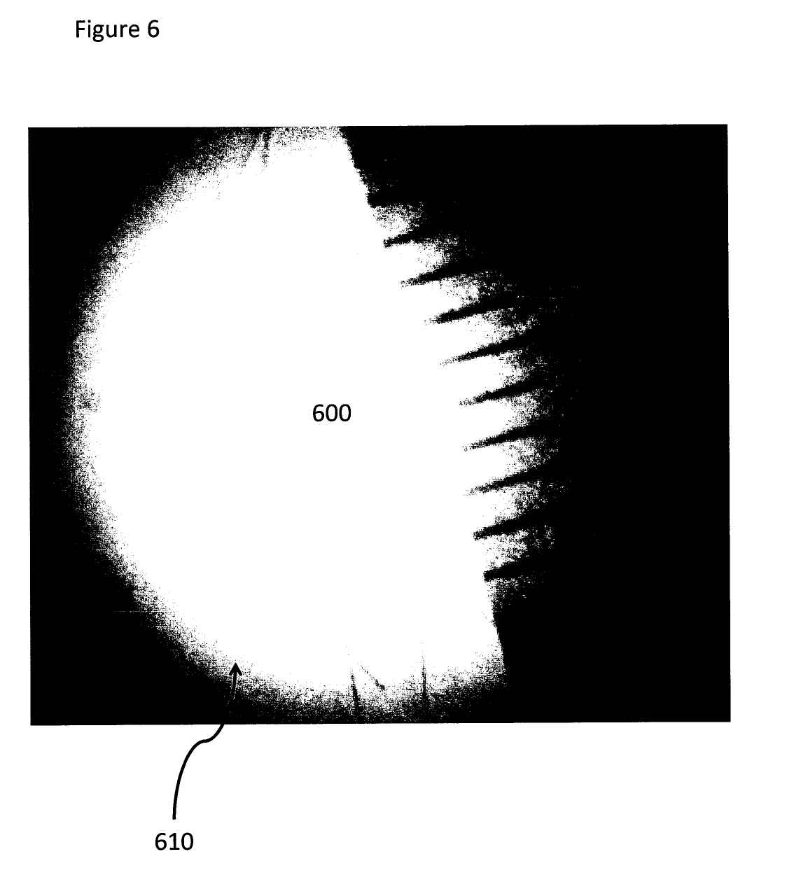

An exemplary imaging geometry optimization is shown in FIG. 6. As illustrated in FIG. 6, the incident beam of light or other electromagnetic radiation can impinge on the body structure, e.g., in the exemplary case, an arm 600. The exemplary beam or light can propagate throughout the tissue and is remitted at a distance from the beam illumination location. The exemplary beam or light that penetrates more deeply into the tissue and that is likely to be more representative of the compartment can be remitted further away from the beam illumination point 600. Accordingly, by measuring the exemplary LSI pattern at a distance away from the beam entry point, such as shown at an exemplary location 610, the exemplary LSI pattern may be more likely to represent the flow distribution of the internal compartment. Increasing the wavelength can further weight the exemplary LSI pattern measurement contribution from deeper within the tissue.

It is also possible to use the exemplary embodiments of the methods and systems according to the present invention for overcoming skin vascular perfusion, including, as shown in FIG. 1, an intermittent tourniquet application 160 that selectively stops skin blood flow while not affecting muscle flow. An additional exemplary embodiment of the system and method according to the present invention can be used to apply a tourniquet using a device that can be transparent and possibly placed substantially over the beam illumination and measurement locations 170.

Another exemplary embodiment of a system and method according to the present invention may be provided that can weight the exemplary LSI pattern measurement to deeper photons that are more likely to have traveled through the compartment is illustrated in FIG. 7. The exemplary system shown in FIG. 7 can include a low coherence light source 700 that may irradiate a sample 720 and a reference arm 710 in, e.g., a Linnik configuration. Light or other electromagnetic radiation from the reference and sample arms can be reflected and detected by cameras 740 and 750 in such a manner that the optical propagation depth over which the speckle pattern is measured may be determined by the path length difference between the reference arm optical path and the sample arm path. In this manner, the coherence gating can provide a probing of the speckle pattern deeper into the tissue, thus possibly increasing the likelihood that the measured speckle pattern comes from the internal body cavity of fascial compartment of interest.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. A system for providing information associated with tissue, comprising:
a first arrangement which is configured to illuminate the tissue with at least one electro-magnetic radiation which is at least one of a coherent light or a partially coherent light;
a second arrangement which is configured to receive the at least one electromagnetic radiation reflected from the tissue and form speckle patterns associated with the at least one electromagnetic radiation; and
a third arrangement which is configured to analyze changes in the speckle patterns at time intervals sufficient to measure motion of or within a fascial compartment of the tissue.

2. The system according to claim 1, wherein the motion includes blood flow.

3. The system according to claim 2, wherein the blood flow is capillary blood flow.

4. The system according to claim 3, wherein the second arrangement is configured to receive the at least one electromagnetic radiation upon an application of pressure to or at a distance from the tissue.

5. The system according to claim 1, wherein the first arrangement is configured to illuminate the tissue at a location position, and the second arrangement is configured to receive the at least one electromagnetic radiation at a second location, and wherein the first and second locations are separated from one another by a predetermined distance.

6. The system according to claim 1, wherein the first, second and third arrangements are provided in a hand-held arrangement.

7. The system according to claim 1, wherein the third arrangement is further configured to measure the speckle patterns at different depths within the sample by moving the reference.

8. The system according to claim 1, wherein the at least one electromagnetic radiation is an interfered radiation from a sample and a reference, and wherein the speckle patterns are associated with the sample.

9. A method for providing information associated with tissue, comprising:
illuminating the tissue with at least one electromagnetic radiation which is at least one of a coherent light or a partially coherent light;
receiving the at least one electromagnetic radiation reflected from the tissue and form speckle patterns associated with the at least one electromagnetic radiation; and
analyzing changes in the speckle patterns at time intervals sufficient to measure motion of or within a fascial compartment of the tissue.

10. A system comprising:
at least one first arrangement which his configured to provide at least one first electro-magnetic radiation to a sample and at least one second electromagnetic radiation to a reference;
a second arrangement which is configured to (i) receive the at least one third electro-magnetic radiation which is an interfered radiation from the sample and the reference and (ii) form speckle patterns associated with the sample; and
a third arrangement which is configured to measure the speckle patterns at different depths within the sample by moving the reference.

11. The system according to claim 10, wherein the third arrangement is further configured to measure motion of or within the sample.

12. The system according to claim 11, wherein the motion is measured within a fascial compartment of the sample.

13. The system according to claim 12, wherein the third arrangement is further configured to analyze changes in the speckle patterns at time intervals sufficient to measure motion of or within a fascial compartment of the tissue.

14. The system according to claim 13, wherein the motion includes blood flow.

15. The system according to claim 14, wherein the blood flow is capillary blood flow.

16. The system according to claim 10, wherein the second arrangement is configured to receive the at least one third electromagnetic radiation upon an application of pressure to or at a distance from the tissue.

17. The system according to claim 10, wherein the first arrangement is configured to illuminate the tissue at a location position, and the second arrangement is configured to receive the at least one third electromagnetic radiation at a second location, and wherein the first and second locations are separated from one another by a predetermined distance.

18. The system according to claim 10, wherein the first, second and third arrangements are provided in a hand-held arrangement.

19. A method comprising:
providing at least one first electromagnetic radiation to a sample and at least one second electromagnetic radiation to a reference;
receiving the at least one third electromagnetic radiation which is an interfered radiation from the sample and the reference;
forming speckle patterns associated with the sample; and
measuring the speckle patterns at different depths within the sample by moving the reference.
